(19) United States
(12) Patent Application Publication
(10) Pub. No.: US 2023/0077273 A1
(43) Pub. Date: Mar. 9, 2023

(54) METHOD AND APPARATUS FOR ASSISTING EXERCISE POSTURE CORRECTION USING WORKING MUSCLE INFORMATION DEPENDING ON MOTION

(71) Applicant: IdeaLink Inc., Seoul (KR)

(72) Inventor: Jong Min Kim, Seoul (KR)

(21) Appl. No.: 17/510,439

(22) Filed: Oct. 26, 2021

(30) Foreign Application Priority Data
Sep. 8, 2021 (KR) 10-2021-0119553

(51) Int. Cl. A63B 24/00; A63B 69/36

(52) U.S. Cl. CPC A63B 24/0006; A63B 24/0062; A63B 69/3623; A63B 2024/0012; A63B 2220/806; A63B 2024/0068; A63B 2024/0071; A63B 2230/62

(57) ABSTRACT

Disclosed is an exercise posture correction assistance method using working muscle information depending on motion performed by at least one server, including acquiring first image information containing movement of a user corresponding to a specific practice motion from a user terminal, deriving skeleton information corresponding to the movement of the user based on the first image information, generating second image information by indicating the movement of the user using a skeleton shape based on the first image information and the skeleton information, deriving muscle information corresponding to the specific practice motion from a database, generating muscle movement image information corresponding to the movement of the user based on the skeleton information and the muscle information, and providing the second image information and the muscle movement image information to the user terminal.

### BACKGROUND OF THE INVENTION

### 1. Field of the Invention

The present invention relates to an exercise posture correction assistance method and apparatus using working muscle information depending on motion, and more particularly to an exercise posture correction assistance method and apparatus using working muscle information depending on a specific motion by analyzing an image of the specific motion of a user.

### 2. Description of the Related Art

A specific sport, exercise, and/or competition requires a specific motion, and capital and effort to prepare an environment for practicing the specific motion along with a long learning time are required to acquire the specific motion.

In addition, as a work is frequently performed in a remote manner, it is difficult to learn a specific motion directly face-to-face with an expert (or professional athlete) of a specific sport, exercise, and/or competition. Accordingly, recently, there has been a need for technology for accurately teaching a specific motion using a remote method while reducing learning time, capital, and effort required for practice of the specific movement for a specific sport, exercise, and/or competition.

### SUMMARY OF THE INVENTION

Therefore, the present invention has been made in view of the above problems, and it is an object of the present invention to provide an exercise posture correction assistance method using working muscle information depending on motion.

It is another object of the present invention to provide an exercise posture correction assistance apparatus using working muscle information depending on motion.

In accordance with an aspect of the present invention, the above and other objects can be accomplished by the provision of an exercise posture correction assistance method using working muscle information depending on motion performed by at least one server, including acquiring first image information containing movement of a user corresponding to a specific practice motion from a user terminal, deriving skeleton information corresponding to the movement of the user based on the first image information, generating second image information by indicating the movement of the user using a skeleton shape based on the first image information and the skeleton information, deriving muscle information corresponding to the specific practice motion from a database, generating muscle movement image information corresponding to the movement of the user based on the skeleton information and the muscle information, and providing the second image information and the muscle movement image information to the user terminal.

The exercise posture correction assistance method may further include deriving at least one measurement value corresponding to at least one measurement item preset for the specific practice motion based on the skeleton information, and providing the at least one measurement value to the user terminal.

The exercise posture correction assistance method may further include providing information on a plurality of sports to the user terminal, and selecting a first sport among the plurality of sports by the user terminal, wherein the specific motion may correspond to one of the plurality of motions performed in the first sport.

The muscle information may include information indicating all muscles in the body into which a joint point related to the skeleton is inserted, and the muscle movement image information is generated by matching the joint point to a joint included in the skeleton information.

The deriving the skeleton information corresponding to the movement of the user based on the first image information includes deriving the skeleton information from the first image information based on a pre-trained skeleton extraction model, and the skeleton extraction model may be trained through machine learning by configuring learning movement image information and learning skeleton information corresponding to the learning movement image information as a training data set.

The exercise posture correction assistance method may further include deriving working muscle information and obstructing muscle information corresponding to the specific practice motion from the database, and providing the working muscle information and the obstructing muscle information to the user terminal, wherein the working muscle information may include information on primary working muscle that is directly used when the specific practice motion is performed, and information on secondary working muscle that is indirectly used when the specific practice motion is performed.

The exercise posture correction assistance method may further include indicating a region corresponding to the working muscle information by a first color and indicating a region corresponding to the obstructing muscle information by a second color in the muscle movement image information.

The exercise posture correction assistance method may further include deriving third image information containing movement of an expert corresponding to the specific practice motion from the database, and providing the second image information and the third image information to the user terminal.

In accordance with another aspect of the present invention, there is provided a server for performing an exercise posture correction assistance method using working muscle information depending on motion, including a processor, and a memory for storing at least one command executed by the processor, wherein the at least one command is executed to acquire first image information containing movement of a user corresponding to a specific practice motion from a user terminal, is executed to derive skeleton information corresponding to the movement of the user based on the first image information, is executed to generate second image information by indicating the movement of the user using a skeleton shape based on the first image information and the skeleton information, is executed to derive muscle information corresponding to the specific practice motion from the memory, is executed to generate muscle movement image information corresponding to the movement of the user based on the skeleton information and the muscle information, and is executed to provide the second image information and the muscle movement image information to the user terminal.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram of an exercise posture correction assistance apparatus according to an embodiment of the present invention;

FIG. 2 is a diagram for explaining an exercise posture correction assistance method according to an embodiment of the present invention;

FIG. 3 is a diagram for explaining muscle information according to an embodiment of the present invention;

FIGS. 4 and 5 are diagrams showing a screen of a user terminal for providing an exercise posture correction assistance method according to an embodiment of the present invention;

FIG. 6 is a diagram for explaining a working muscle and an obstructing muscle according to an embodiment of the present invention;

FIG. 7 is a diagram showing a screen of a user terminal for providing image information containing movement of an expert according to an embodiment of the present invention;

FIG. 8 is a diagram showing a screen of user terminal for providing a posture correction method according to an embodiment of the present invention; and

FIG. 9 is a flowchart of an exercise posture correction assistance method according to an embodiment of the present invention.

### DETAILED DESCRIPTION OF THE INVENTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. Like reference numerals refer to like elements throughout.

Terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the teachings of the present invention. The term “and/or” includes any and all combinations of one or more of the associated listed items.

It will be understood that when an element, such as a layer, a region, or a substrate, is referred to as being “on”, “connected to” or “coupled to” another element, it may be directly on, connected or coupled to the other element or intervening elements may be present. In contrast, when an element is referred to as being “directly on,” “directly connected to” or “directly coupled to” another element or layer, there are no intervening elements or layers present.

The terms used in the present specification are used for explaining a specific exemplary embodiment, not limiting the present invention. Thus, the singular expressions in the present specification include the plural expressions unless clearly specified otherwise in context. Also, terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Reference will now be made in detail to embodiments, examples of which are illustrated in the accompanying drawings. In describing the present invention, in order to facilitate the overall understanding, the same reference numerals are used for the same components in the drawings, and repeated description of the same components is omitted.

FIG. 1 is a block diagram of an exercise posture correction assistance apparatus according to an embodiment of the present invention.

Referring to FIG. 1, an exercise posture correction assistance apparatus 100 according to an embodiment of the present invention may include at least one processor 110 and a memory 120 and may further include at least one of a transceiving device 130, an input interface device 140, an output interface device 150, or a storage device 160. Components included in the exercise posture correction assistance apparatus 100 may be connected to each other by a bus 170 and may communicate therewith.

For example, the at least one processor 110 may refer to a central processing unit (CPU), a graphics processing unit (GPU), or a dedicated processor that performs methods according to embodiments of the present invention. Each of the memory 120 and the storage device 160 may be configured as at least one of a volatile storage medium and a non-volatile storage medium. For example, the memory 120 may be one of a read only memory (ROM) and a random access memory (RAM), and the storage device 160 may be flash memory, hard disk drive (HDD), a solid state drive (SSD), or various memory cards (e.g., a micro SD card).

For example, the memory 120 or the storage device 160 may store at least one command to be executable by the at least one processor 110. For example, at least one command may include a command for acquiring first image information containing movement of a user corresponding to a specific practice motion from a user terminal, a command for deriving skeleton information corresponding to the movement of the user based on the first image information, a command for generating second image information by indicating the movement of the user using a skeleton shape based on the first image information and the skeleton information, a command for deriving muscle information corresponding to the specific practice motion from a database, a command for generating muscle movement image information corresponding to the movement of the user based on the skeleton information and the muscle information, and a command for providing the second image information and the muscle movement image information to the user terminal.

For example, at least one command may further include a command for deriving at least one measurement value corresponding to at least one measurement item preset for the specific practice motion based on the skeleton information, and a command for providing the at least one measurement value to the user terminal.

For example, at least one command may further include a command for providing information on a plurality of sports to the user terminal, and a command for selecting a first sport among the plurality of sports by the user terminal, and the specific motion may correspond to one of the plurality of motions performed in the first sport.

For example, the muscle information may include information indicating all muscles in the body into which a joint point related to the skeleton is inserted, and the muscle movement image information may be generated by matching the joint point to a joint included in the skeleton information.

For example, the command for deriving skeleton information corresponding to the movement of the user based on the first image information may include a command for deriving the skeleton information from the first image information based on a pre-trained skeleton extraction model, and the skeleton extraction model may be trained through machine learning by configuring learning movement image information and learning skeleton information corresponding to the learning movement image information as a training data set.

For example, at least one command may further include a command for deriving working muscle information and obstructing muscle information corresponding to the specific practice motion from the database, and a command for providing the working muscle information and the obstructing muscle information to the user terminal, and the working muscle information may include information on primary working muscle that is directly used when the specific practice motion is performed, and information on secondary working muscle that is indirectly used when the specific practice motion is performed.

For example, at least one command may further include a command for indicating a region corresponding to the working muscle information by a first color and indicating a region corresponding to the obstructing muscle information by a second color in the muscle movement image information.

For example, at least one command may further include a command for deriving third image information containing movement of an expert corresponding to the specific practice motion from the database, and a command for providing the second image information and the third image information to the user terminal.

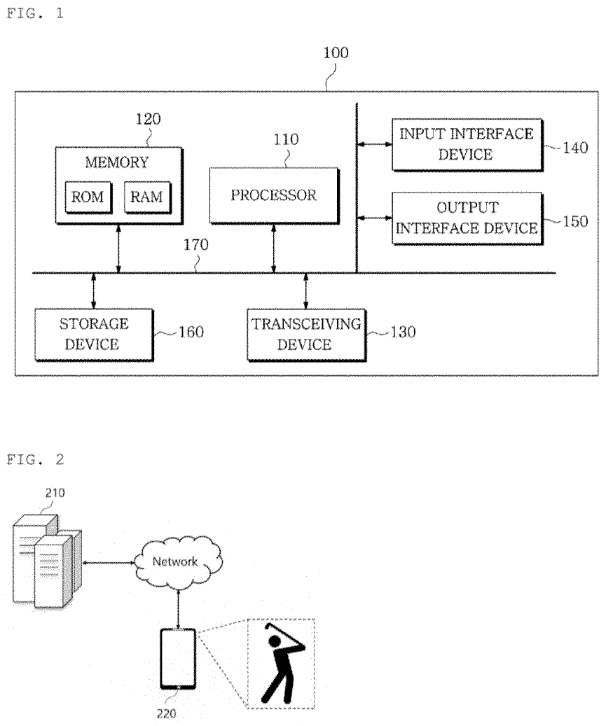

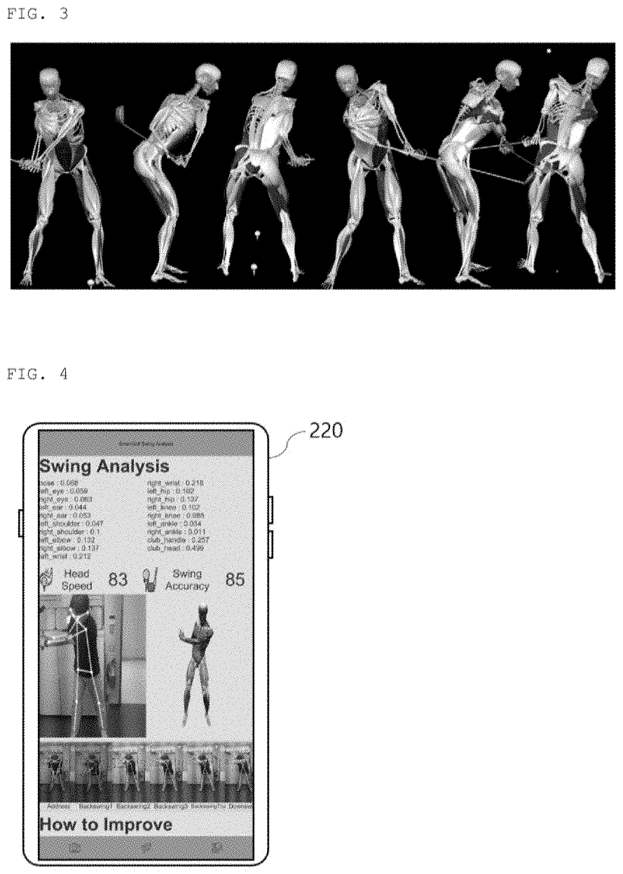

FIG. 2 is a diagram for explaining an exercise posture correction assistance method according to an embodiment of the present invention.

Referring to FIG. 2, the exercise posture correction assistance method according to an embodiment of the present invention may be performed through wired or wireless communication between an exercise posture correction assisting server (hereinafter, a server) 210 and a user terminal 220. Here, the exercise posture correction assisting server 210 may refer to the aforementioned exercise posture correction assistance apparatus.

The user terminal 220 may refer to an electronic device capable of communicating and computing, and for example, may be a communicable desktop computer, a laptop computer, a notebook computer, a smart phone, a tablet. PC, a mobile phone, a smart watch, smart glasses, an e-book reader, a portable multimedia player (PMP), a portable game console, a navigation device, a digital camera, a digital multimedia broadcasting (DMB) player, a digital audio recorder, a digital audio player, a digital video recorder, a digital video player, a personal digital assistant (PDA), or the like.

The user terminal 220 may be an electronic device including a photographing device installed therein for capturing an image of the movement of the user, but the user terminal 220 is not limited to the electronic device including the photographing device installed therein because it is possible to also receive the image of the movement of the user through a network.

FIG. 3 is a diagram for explaining muscle information according to an embodiment of the present invention.

It would be obvious that, when a person moves, he or she may use at least one muscle of the body, and when a specific motion is repeated in the same way, the same muscle works in the same way. That is, when the person follows a certain motion, he or she actually follows action of muscle that performs the certain motion.

Referring to FIG. 3 in which a swing motion of golf is exemplified, the swing motion always needs the same movement, and thus when an accurate swing motion is performed, muscles that directly or indirectly work may be determined, and the same muscles may always be used.

An embodiment of the present invention may be to improve learning efficiency of a user by visually providing these working muscles to the user. That is, people may or may not apply force consciously or directly to all muscles, but when recognizing a specific part of muscle for achieving a correct posture, they may learn the correct posture more rapidly, which is a main objective of an embodiment.

Hereinafter, motion according to an embodiment will be described.

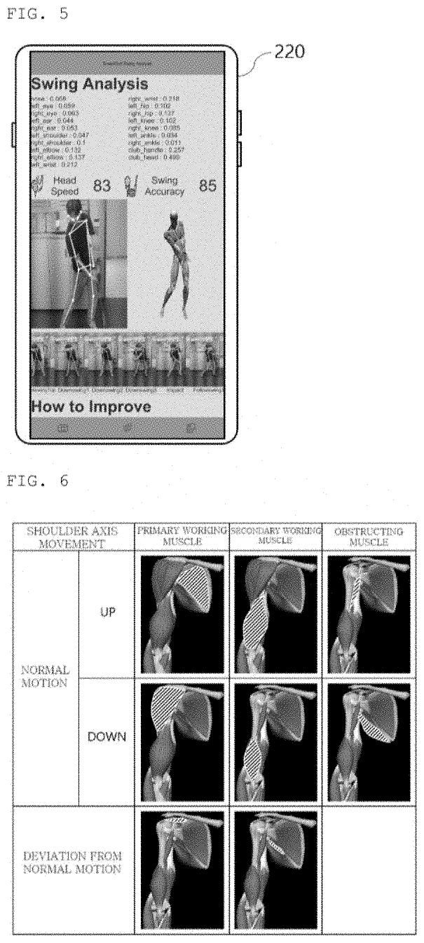

FIGS. 4 and 5 are diagrams showing a screen of a user terminal for providing an exercise posture correction assistance method according to an embodiment of the present invention.

Although the exercise posture correction assistance method according to an embodiment of the present invention is described using golf as an example, the present invention may be applied to other sports in the same way. Thus, only motions of golf are illustrated in the drawings, but the present invention is not limited thereto.

Accordingly, according to an embodiment, information on a plurality of sports may be provided to a user, and the user may select a first sport among a plurality of sports through the user terminal. Subsequently, according to an embodiment, information on a plurality of motions included in the first sport may be provided to the user terminal, and the user may select a specific practice motion among the plurality of motions through the user terminal.

After the specific practice motion to be practiced by the user is selected, the user may acquire first image information including the movement of the user corresponding to the specific practice motion through the user terminal. Here, the first image information may be acquired through a photographing device installed in the user terminal, but may also be acquired from another external server or another user terminal through a network.

Then, the server may derive skeleton information corresponding to the movement of the user based on the first image information. Here, the skeleton information may include information on a joint, indicating a joint part or an edge part, of the body of the user and information on a line between joints. For example, the joint part or the edge part corresponding to the joint may include at least one of nose, left_eye, right_eye, left_ear, right_ear, left_shoulder, right_shouider, ieft_elbow, right_elbow, left_wrist, right_wrist, left_hip, right_hip, left_knee, right_knee, left_ankle, right_ankle, club_handle, or club_head.

The skeleton information may be derived using the pre-trained skeleton extraction model. That is, the server may derive the skeleton information from the first image information based on the pre-trained skeleton extraction model. Here, the skeleton extraction model may be pre-trained through machine learning by configuring learning movement image information and learning skeleton information corresponding to the learning movement image information as a training data sot, and an algorithm used in machine learning may be a deep neural network (DNN), a convolutional neural network (CNN), a recurrent neural network (FINN), a restricted Boltzmann machine (RBM), or a deep belief network (DBN), but other algorithms more suitable for learning may also be used. Here, each of the algorithms is a well-known technology, a description thereof is omitted.

The server may generate second image information by overlapping the derived skeleton information on the first image information. That is, the second image information may be information on an image formed by indicating a joint and a line based on the skeleton information on the movement of the user. For example, referring to FIGS. 4 and 5, the image based on the second image information may be an image positioned at a left center of the screen of the user terminal.

The server may derive at least one measurement value corresponding to at least one first measurement item preset for the specific practice motion based on the skeleton information. Referring to FIGS. 4 and 5, at least one first measurement item may include at least one of head speed or swing accuracy, and the measurement value of the first measurement item may be derived at one of natural numbers between 0 and 100. For example, at least one first measurement item may be derived based on at least one of location-related measurement information of a club handle (club_handle) or location-related measurement information of a club head (club_head).

In addition, the server may derive muscle information corresponding to the specific practice motion from a database and may derive at least one measurement value corresponding to at least one second measurement item preset based on the muscle information and the skeleton information. Referring to FIGS. 4 and 5, at least one second measurement item may include at least one of location-related measurement information of nose (nose), location-related measurement information of left eye (left_eye), location-related measurement information of right eye (right_eye), location-related measurement information of left ear (left_ear), location-related measurement information of right ear (right_ear), location-related measurement information of left shoulder (left_shoulder), location-related measurement information on right shoulder (right_shoulder), location-related measurement information of left elbow (left_elbow), location-related measurement information of right elbow (right_elbow), location-related measurement information of left wrist (left_wrist), location-related measurement information of right wrist (right_wrist), location-related measurement information of left hip (left_hip), location-related measurement information of right hip (right_hip), location-related measurement information of left knee (left_knee), location-related measurement information of right knee (right_knee), location-related measurement information of left ankle (left_ankle), location-related measurement information of right ankle (right_ankle), location-related measurement information of a club handle (club_handle), or location-related measurement information of a club head (club_head).

For example, the muscle information may be information indicating muscle in which a joint point related to skeleton is inserted, and each piece of location-related measurement information may also indicate information on a distance difference between each location in the movement of the user in an image and each location of a joint point included in the muscle information. Through the information, the user may check a part that makes the greatest difference. In order to accurately derive the difference, the server may first acquire height information of the user and image information of a ready posture to generate personal information of the user and may correct the muscle information in consideration of the generated personal information.

The server may provide the measurement value as derived above with the second image information to the user terminal as shown in FIGS. 4 and 5.

The server may also generate muscle movement image information corresponding to the movement of the user based on the skeleton information and the muscle information. That is, the server may generate a muscle image based on information on an image of muscle movement that is the same as the movement of the user and may provide the muscle image with the second image information and the measurement value to the user terminal as shown in FIGS. 4 and 5. Referring to FIGS. 4 and 5, the image of the muscle movement image information may indicate an image positioned at a right center of the screen of the user terminal. Here, the muscle movement image information may be generated by matching a joint point included in the muscle information to a joint included in the skeleton information, but may be generated using various other methods, and thus the present invention is not limited thereto.

For example, the server may provide a video image based on the second image information and the muscle movement image information to the user terminal, but may also provide a plurality of still images to the user terminal.

When the second image information and the muscle movement image information are provided as a plurality of still images, an image based on the first image information may be first split into a plurality of still images at a preset time interval, and motions according to the above embodiment may be performed on each of the split still images. In addition, the plurality of still images may be displayed at a bottom of the screen of the user terminal as shown in FIGS. 4 and 5.

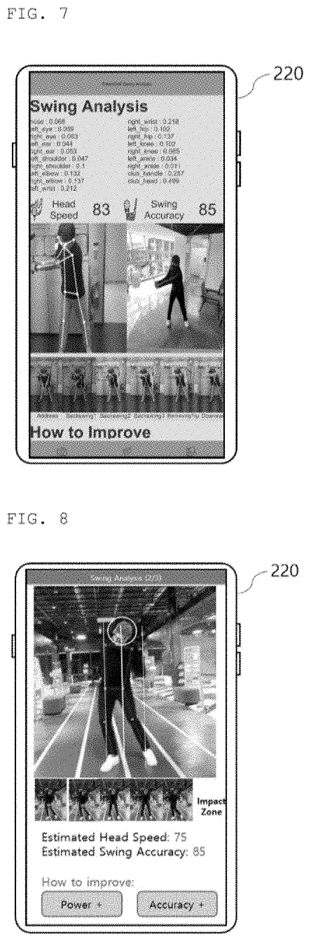

FIG. 6 is a diagram for explaining a working muscle and an obstructing muscle according to an embodiment of the present invention.

The server may provide not only the muscle movement based on the movement of the user but also working muscle that works and obstructing muscle that obstructs during the specific practice motion to the user, and information thereon may be pre-stored in the database.

For example, when shoulder axis movement is performed, the working muscle and the obstructing muscle may be muscle of a region indicated by diagonal lines, and visual information about the muscle may be provided to a user through a user terminal. For example, when the specific practice motion is a swing motion of golf, working muscle and obstructing muscle used in a region UP in which a club is raised and working muscle and obstructing muscle used in a region DOWN in which the club is lowered may be provided depending on a swing motion. In addition, information on muscle that works when motion is outside a normal motion range (deviation from normal motion) may also be provided.

The working muscle may be divided into primary working muscle that directly works in the corresponding motion and secondary working muscle that indirectly works in the motion and may be provided.

For example, after deriving the working muscle information and the obstructing muscle information from the database, the server may indicate a region corresponding to the working muscle information and a region corresponding to the obstructing muscle information on the generated muscle movement image information and may provide the information. In this case, the region corresponding to the working muscle information may be indicated by the first color, the region corresponding to the obstructing muscle information may be indicated with the second color, and the first color and the second color may be different colors.

FIG. 7 is a diagram showing a screen of a user terminal for providing image information containing movement of an expert according to an embodiment of the present invention.

Referring to FIG. 7, a server according to an embodiment may derive third image information including the movement of the expert for the specific practice motion in addition to the muscle movement image information from a database and may also provide the information to the user.

When the third image information is provided, the movement of the user and the movement of the expert may also be provided side by side as shown in FIG. 7 to compare the movement of the user and the movement of the expert. That is, the server may provide the second image information and the third image information side by side to the user terminal.

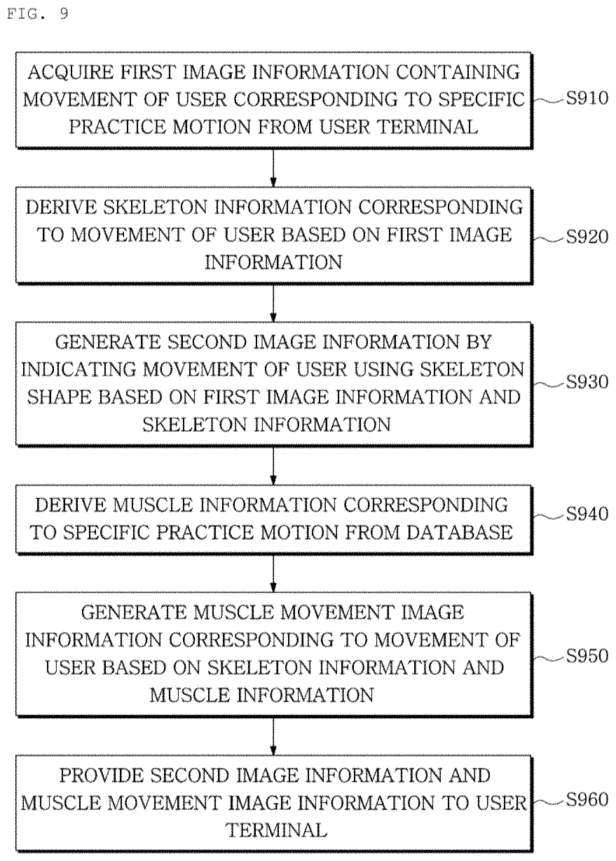

FIG. 8 is a diagram showing a screen of user terminal for providing a posture correction method according to an embodiment of the present invention.

Referring to FIG. 8, a server according to an embodiment may provide not only the second image information but also information on a method of improving power and information on a method of improving accuracy. Here, the method of improving power may be provided when the user selects a region Power+ at a bottom of the screen of the user terminal, and the method of improving accuracy may be provided when the user selects a region Accuracy+ at the bottom of the screen of the user terminal.

For example, the information on the method of improving power and/or the information on the method of improving accuracy may be derived and provided based on a measurement item equal to or greater than a threshold value among measurement values of at least one piece of second measurement item, and when there is no measurement item equal to or greater than the threshold value, the measurement value may be derived and provided based on the highest measurement item.

For example, after the second image information is provided to a plurality of preregistered expert terminals, power-related or accuracy-related comment information for the second image information may be acquired from the plurality of expert terminals and may be provided to the user terminal. Here, the number of the plurality of experts may be determined based on a measurement value of high speed or swing accuracy as the first measurement item. That is, when the information on the method of improving power is selected, the number of the plurality of experts may be determined based on the measurement value of the head speed, and when the information on the method of improving accuracy is selected, the number of the plurality of experts may be determined based on the measurement value of the swing accuracy. The number of the plurality of experts may be increased as the measurement value increases. This is because an improvement direction is clearly checked only using comments of some experts for a beginner with a low measurement value but there are various comments and various improvement directions depending on experts for an advanced user with a high measurement value, and thus many opinions need to be transferred to the user. However, the number of the plurality of experts may be at least two.

FIG. 9 is a flowchart of an exercise posture correction assistance method according to an embodiment of the present invention.

Referring to FIG. 9, in operation S910 according to an embodiment, first image information containing movement of a user corresponding to specific practice motion may be acquired from a user terminal. Here, the specific motion may correspond to one of a plurality of motions performed in a first sport that is selected among a plurality of sports by the user.

In operation S920 according to an embodiment, skeleton information corresponding to the movement of the user may be derived based on the first image information. Here, the skeleton information may be derived from the first image information based on a pre-trained skeleton extraction model, and the skeleton extraction model may be trained through machine learning by configuring learning movement image information and learning skeleton information corresponding to the learning movement image information as a training data set.

In operation S930 according to an embodiment, second image information may be generated by indicating the movement of the user using a skeleton shape based on the first image information and the skeleton information.

In operation S940 according to an embodiment, muscle information corresponding to the specific practice motion may be derived from a database. For example, the muscle information may include information indicating all muscles in the body into which a joint point related to the skeleton is inserted.

In operation S950 according to an embodiment, muscle movement image information corresponding to the movement of the user may be generated based on the skeleton information and the muscle information. For example, the muscle movement image information may be generated by matching the joint point to a joint included in the skeleton information.

Operation S960 according to an embodiment may include an operation of providing the second image information and the muscle movement image information to the user terminal.

Although not shown in FIG. 9, according to an embodiment, at least one measurement value corresponding to at least one measurement item preset for the specific practice motion may be derived based on the skeleton information, and at least one measurement value may also be provided to the user terminal.

According to an embodiment, working muscle information and obstructing muscle information corresponding to the specific practice motion may be derived from a database, and the working muscle information and the obstructing muscle information may also be provided to the user terminal. Here, the working muscle information may include information on primary working muscle that is directly used when the specific practice motion is performed and information on secondary working muscle that is indirectly used when the specific practice motion is performed.

According to an embodiment, in the muscle movement image information, a region corresponding to the working muscle information may be indicated by a first color, and a region corresponding to the obstructing muscle information may be indicated by a second color.

For example, according to an embodiment, the third image information containing the movement of the expert corresponding to the specific practice motion may be derived from a database, and the second image information and the third image information may also be provided to the user terminal.

The operations according to an embodiment of the present invention may also be embodied as computer readable program or code on a computer readable recording medium. The computer readable recording medium is any data storage device that can store programs or data which can be thereafter read by a computer system. The computer readable recording medium can also be distributed over network coupled computer systems so that the computer readable code is stored and executed in a distributed fashion.

When an embodiment is implemented in software, the aforementioned scheme may be implemented as a module (a process or a function) that performs the aforementioned function. The module may be stored in a memory and may be executed by a processor. The memory may be positioned inside or outside the processor and may be connected to the processor using various well-known devices.

The computer readable recording medium may include a hardware device that is particularly configured to store and execute a program command, such as ROM, RAM, or flash memory.

The program command may include machine language code created by a compiler and high-level language code executable by a computer using an interpreter and the like.

Although some aspects of the invention have been described in terms of an apparatus, the aspects may also be represented through a description of a corresponding method, and here, a block or an apparatus may correspond to an operation of a method or the feature of an operation of a method. Similarly, aspects described in terms of a method may also be represented by a corresponding block or item or the feature of a corresponding apparatus. Some or all of operations of a method may be performed by (or using) a hardware device such as, for example, a microprocessor, a programmable computer, or an electronic circuit. In some embodiments, one or more of the most important operations of a method may be performed such a device.

According to embodiments, a programmable logic device (e.g., a field programmable gate array) may be used to perform some or ail of functions of the methods described herein. According to embodiments, the field programmable gate array may be operated with a microprocessor for performing one of the methods described herein. In general, methods may be performed by any hardware device.

According to the present invention, a posture correction effect may be maximized by checking muscle that works and muscle that obstructs a specific motion when the specific motion is performed.

According to the present invention, muscle-based information may be provided to notify a user of which muscle the user needs to care about and perform motion.

It will be appreciated by persons skilled in the art that the effects that could be achieved with the present invention are not limited to what has been particularly described hereinabove and other advantages of the present invention will be more clearly understood from the above detailed description.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An exercise posture correction assistance method using working muscle information depending on motion performed by at least one server, comprising:
acquiring first image information containing movement of a user corresponding to a specific practice motion from a user terminal;
deriving skeleton information corresponding to the movement of the user based on the first image information;
generating second image information by indicating the movement of the user using a skeleton shape based on the first image information and the skeleton information;
deriving muscle information corresponding to the specific practice motion from a database;
generating muscle movement image information corresponding to the movement of the user based on the skeleton information and the muscle information; and
providing the second image information and the muscle movement image information to the user terminal.

2. The exercise posture correction assistance method according to claim 1, further comprising:
deriving at least one measurement value corresponding to at least one measurement item preset for the specific practice motion based on the skeleton information; and
providing the at least one measurement value to the user terminal.

3. The exercise posture correction assistance method according to claim 1, further comprising:
providing information on a plurality of sports to the user terminal; and
selecting a first sport among the plurality of sports by the user terminal,
wherein the specific motion corresponds to one of the plurality of motions performed in the first sport.

4. The exercise posture correction assistance method according to claim 1, wherein the muscle information includes information indicating all muscles in the body into which a joint point related to the skeleton is inserted, and the muscle movement image information is generated by matching the joint point to a joint included in the skeleton information.

5. The exercise posture correction assistance method according to claim 1, wherein:
the deriving the skeleton information corresponding to the movement of the user based on the first image information includes deriving the skeleton information from the first image information based on a pre-trained skeleton extraction model; and
the skeleton extraction model is trained through machine learning by configuring learning movement image information and learning skeleton information corresponding to the learning movement image information as a training data set.

6. The exercise posture correction assistance method according to claim 1, further comprising:
deriving working muscle information and obstructing muscle information corresponding to the specific practice motion from the database; and
providing the working muscle information and the obstructing muscle information to the user terminal,
wherein the working muscle information includes information on primary working muscle that is directly used when the specific practice motion is performed, and information on secondary working muscle that is indirectly used when the specific practice motion is performed.

7. The exercise posture correction assistance method according to claim 6, further comprising:
indicating a region corresponding to the working muscle information by a first color and indicating a region corresponding to the obstructing muscle information by a second color in the muscle movement image information.

8. The exercise posture correction assistance method according to claim 1, further comprising:
deriving third image information containing movement of an expert corresponding to the specific practice motion from the database; and
providing the second image information and the third image information to the user terminal.
